(19) United States
(12) Patent Application Publication
(10) Pub. No.: US 2013/0261146 A1
(43) Pub. Date: Oct. 3, 2013

(54) NITRIC OXIDE AND ITS BIOMEDICAL SIGNIFICANCE

(71) Applicants: George B. Stefano, Melville, NY (US); Wei Zhu, Babylon, NY (US); Kirk Mantione, Seaford, NY (US); Richard M. Kream, Huntington, NY (US)

(72) Inventors: George B. Stefano, Melville, NY (US); Wei Zhu, Babylon, NY (US); Kirk Mantione, Seaford, NY (US); Richard M. Kream, Huntington, NY (US)

(73) Assignee: The Research Foundation of State University of New York, Albany, NY (US)

(21) Appl. No.: 13/992,597

(22) PCT Filed: Dec. 9, 2010
(86) PCT No.: PCT/US2010/059630

§ 371 Date: Jun. 7, 2013

(51) Int. Cl. C07C 251/24; C07C 43/275; C07D 221/18; C07C 59/42

(52) U.S. Cl. CPC C07C 251/24; C07C 59/42; C07C 43/275; C07D 221/18
USPC 514/284; 554/223; 514/549; 568/636; 514/721; 546/75; 564/274; 514/641

(57) ABSTRACT

A pharmaceutical composition for stimulating nitric oxide production in mammalian cells, the pharmaceutical composition including at least one compound selected from a group consisting of: 2,3-dihydroxypropyl oleate; bis(m-phenoxyphenyl) ether; 6-acetyl-5,6,6a,7-tetrahydro-4H-dibezo(de,g)quinoline; and (+)-N-(p-(2-methylbutoxy)benzylidene)-4-(2-methylbutyl)aniline.

### REFERENCE TO PRIOR APPLICATION

This application claims the benefit of co-pending U.S. application Ser. No. 10/526,091 filed on Aug. 15, 2005 and is incorporated by reference.

### INVENTION FIELD

The invention relates generally to pharmaceutical compositions and methods of treatment and more specifically to pharmaceutical compositions for stimulating nitric oxide (NO) release.

### INVENTION BACKGROUND

Nitric oxide (NO) is a major signaling molecule in the mammalian immune, cardiovascular and nervous systems. NO produced at one site can have an effect on tissues at a distance. NO is produced from L-arginine by the enzyme, nitric oxide synthase (NOS). NOS occurs in three forms: endothelial (e), neuronal (n), and inducible (i) NOS. The first two forms are constitutively expressed and Ca²⁺ dependent. Inducible (i) NOS is Ca²⁺ independent. The three forms of NOS are encoded for on three distinct genes on chromosomes, 7, 12, and 17, respectively. In general, n- and e-NOS depend on intracellular calcium transients and release NO in the nM range, whereas iNOS, following an induction/latency period, can release NO in the μM range for extended periods of time. The presence of constitutive and inducible forms of NOS suggest that they may have distinct functions.

c- and i-NOS can be distinguished on the basis of the length of time necessary to see an increase in levels of NO and the length of time these elevated levels can be maintained. NO derived from cNOS may occur in two functional forms: the first is always present at low “tonal” or “basal” levels; this basal level can be slightly increased for a short time in response to certain signals, e.g., acetylcholine (ACH). This brief enhanced release of cNOS derived NO can have profound physiological actions, which are evident long after NO has returned to its basal level, for a longer period of time. For example, endothelial cells briefly exposed to morphine and eNOS change their shape from elongated to round, a process that takes several hours.

iNOS is induced by various signal molecules, e.g., proinflammatory cytokines. The induction of i-NOS is usually seen after a 3-4 hour delay; iNOS is capable of producing NO for 24-48 hours. These data suggest that NO is always present and that the levels of NO can be regulated either rapidly or slowly depending on the organism's needs. The presence of different regulatory processes implies that NO has different functions, and/or that the levels of NO must be progressively increased in order for it to exert its function.

NO functions as a vascular, immune and neural signal molecule and also has general antibacterial, antiviral actions and the ability to down-regulate proinflammatory events. In the vascular and immune systems, one of the key stages in the immune response is the recruitment and activation of leukocytes by the endothelium. Leukocyte activation by the endothelium occurs in stages. The initial step is the attraction of the leukocytes to the endothelium. This is followed by increased leukocyte adhesion and change in shape and finally migration across the endothelium. These cellular changes are accompanied by scheduled changes in synthesis of molecules that regulate cell-matrix interactions.

Normally, non-activated leukocytes roll along the endothelium. The interaction between the two cell types is loose and reversible and mediated by a family of adhesion molecules known as selectins. Activation of leukocytes occurs in response to the release of several chemoattractants including leukotriene B₄ and interleukin 8 (IL-8). In the presence of these agents, immunocytes cease to roll, becoming “activated”: they start to flatten and adhere with greater strength to the endothelial lining. Activation is mediated by a family of adhesion molecules call the integrins, such as ICAM-1 and VCAM-1. Adherent immunocytes are able to undergo transendothelial migration in the presence of PECAM-1. This immunocyte-endothelial interaction is down-regulated by NO. NO inhibits platelet and neutrophil aggregation and can diminish the adherence and level of activation of leukocytes and endothelial cells. NOS inhibitors increase platelet adhesion and enhance leukocyte adhesion. NO plays a similar role involving the microglia cells of the nervous system's immune response.

The central nervous system (CNS) is unique in that it uses all three isoforms of NOS to produce NO. The constitutive isoforms e- and n-NOS are found in the normal CNS; however, iNOS is not expressed in the healthy CNS. Pathological states, e.g., trama, cerebral ischemia and neuronal diseases, increase the levels of e- and nNOS and induce iNOS activity. cNOS derived NO has the ability to down-regulate proinflammatory events via inhibition of NF-κB activation of proinflammatory cytokines.

NO upregulates several enzymes involved in immunoregulation, including neutral endopeptidese 24.11 (CALLA, acute lymphoblastic leukemic antigen, enkephalinase) or CD10. Thus, cNOS derived NO stimulates enzymes that process protein gene products, implying a link between signaling processes involving NO and naturally occurring antibacterial peptides. NO controls and regulates enzymes that are responsible for liberating these crucial molecules that have a proactive protective function.

Evidence has also been provided that NO plays a role in neurotransmitter release. Morphine and cNOS derived NO release growth hormone and ACTH from rat brain fragments; these neuropeptides are involved in the stress response. Thus, NO is involved in vasodilation, antibacterial and antiviral responses, signal molecule release and inhibition of immunocyte adherence to the endothelium.

There appears to be a tonal or basal level of NO that is physiologically significant. Endothelia from non-insulin dependent diabetics do not exhibit a tonal level of NO and in these individuals vascular disease causes disability and eventual death. A number of researchers have attributed vascular disease in part to alterations associated with eNOS-derived NO and some have speculated this may be due to enhanced free radical generation. Decreases in basal NO levels may also contribute to enhanced platelet function and various neuropathies.

Thus, it appears that tonal or basal NO levels are important in limiting the degree of excitation of nervous, immune and vascular tissues. This tonal NO may manifest itself via effects on adhesion-mediated processes via NF-κB. Estrogen may exert its beneficial vascular protective actions via these processes as well, since it also releases cNOS derived NO. Strengthening this hypothesis is the finding of the cannabinoid CB1 receptor type on mammalian endothelial cells and the finding of a mu opiate receptor on human vascular endothelial cells. (Three general classes of cell surface opioid receptors (kappa, delta and mu) have been described. Receptors exhibiting high binding specificity for morphine have been designated mu opioid receptors.) Detailed analysis has revealed the existence of multiple mu opioid receptor subtypes. Isolated nucleic acid sequences encoding various mu receptors and polypeptides comprising mu receptors (and referred to here as “mu3 opioid receptor(s)”) are disclosed in detail in PCT Patent Publication WO 99/24471, published 20 May 1999. See also, Molecular Identification and Functional Expression of μ₃, a Novel Alternatively Spliced Variant of the Human μ Opiate Receptor Gene.

Consequently, promoting NO generation at normal or slightly enhanced levels may have significant health value. While the health promoting effects of many plants are well known, how and why this occurs at a molecular level is less understood. See Stefano and Miller, Communication between animal cells and the plant foods they ingest: Phyto-zooidal dependencies and signaling (Review), Intl J Mol Medicine 10: 413-21 (2002) incorporated by reference herein.

### INVENTION SUMMARY

A first aspect of the invention is a pharmaceutical composition for stimulating nitric oxide production in mammalian cells, the pharmaceutical composition comprising: at least one compound selected from a group consisting of: 2,3-dihydroxypropyl oleate; bis(m-phenoxyphenyl)ether; 6-acetyl-5,6,6a,7-tetrahydro-4H-dibezo(de,g)quinoline; and (+)-N-(p-(2-methylbutoxy)benzylidene)-4-(2-methylbutyl)aniline.

A second aspect of the invention is a method of stimulating nitric oxide production in an individual in need of such treatment, the method comprising: administering to the individual at least one compound selected from a group consisting of: 2,3-dihydroxypropyl oleate; bis(m-phenoxyphenyl)ether; 6-acetyl-5,6,6a,7-tetrahydro-4H-dibezo(de,g)quinoline; and (+)-N-(p-(2-methylbutoxy)benzylidene)-4-(2-methylbutyl)aniline.

Other features and advantages will be apparent from the following detailed description, drawings and claims.

### DRAWING DESCRIPTIONS

These and other features of this invention will be more readily understood from the following detailed description of the various aspects of the invention taken in conjunction with the accompanying drawings that depict various embodiments of the invention, in which:

FIG. 1 illustrates mass spectrometry results for Healthin 1.

FIG. 2 illustrates mass spectrometry results for Healthin 2.

FIG. 3 illustrates compound characteristics for 2,3-dihydroxypropyl oleate.

FIG. 4 illustrates mass spectrometry results for 2,3-dihydroxypropyl oleate.

FIG. 5 illustrates comparative mass spectrometry analysis for 2,3-dihydroxypropyl oleate and Healthin 1.

FIG. 6 illustrates comparative mass spectrometry analysis for 2,3-dihydroxypropyl oleate and Healthin 2.

FIG. 7 illustrates compound characteristics for bis(m-phenoxyphenyl)ether.

FIG. 8 illustrates mass spectrometry results for bis(m-phenoxyphenyl)ether.

FIG. 9 illustrates comparative mass spectrometry analysis for bis(m-phenoxyphenyl)ether and Healthin 1.

FIG. 10 illustrates comparative mass spectrometry analysis for bis(m-phenoxyphenyl)ether and Healthin 2.

FIG. 11 illustrates compound characteristics for 6-acetyl-5,6,6a,7-tetrahydro-4H-dibezo(de,g)quinoline.

FIG. 12 illustrates mass spectrometry results for 6-acetyl-5,6,6a,7-tetrahydro-4H-dibezo(de,g)quinoline.

FIG. 13 illustrates comparative mass spectrometry analysis for 6-acetyl-5,6,6a,7-tetrahydro-4H-dibezo(de,g)quinoline and Healthin 1.

FIG. 14 illustrates comparative mass spectrometry analysis for 6-acetyl-5,6,6a,7-tetrahydro-4H-dibezo(de,g)quinoline and Healthin 2.

FIG. 15 illustrates compound characteristics for (+)-N-(p-(2-methylbutoxy)benzylidene)-4-(2-methylbutyl)aniline.

FIG. 16 illustrates mass spectrometry results for (+)-N-(p-(2-methylbutoxy)benzylidene)-4-(2-methylbutyl)aniline.

FIG. 17 illustrates comparative mass spectrometry analysis for (+)-N-(p-(2-methylbutoxy)benzylidene)-4-(2-methylbutyl)aniline and Healthin 2.

FIG. 18 illustrates the HPLC chromatogram of the wheat grass extraction detailed in Example 1.

FIG. 19 illustrates the HPLC chromatogram of the white willow bark extraction detailed in Example 2.

FIG. 20 illustrates the mass spectrometric analysis detailed in Example 3.

FIG. 21 illustrates the mass spectrometric analysis detailed in Example 4.

FIGS. 22 and 23 illustrate the results of the pedal ganglia and endothelial cell stimulation by Agropyrum spp. plant extracts as detailed in Example 5.

FIGS. 24 and 25 illustrate the results of the pedal ganglia and endothelial cell stimulation by Salix alba extracts as detailed in Example 6.

FIG. 26 illustrates the results of the pedal ganglia cell stimulation by Taracum officinale extracts as detailed in Example 7.

FIG. 27 illustrates the results of the pedal ganglia cell stimulation by Vitus extracts as detailed in Example 8.

FIG. 28 illustrates real-time evoked release of NO from pooled M. edulis pedal ganglia by a white willow bark lipid extract in comparison to cold and boiling water white willow bark water extracts in Example 11.

FIG. 29 illustrates a dose response relationship of lipid extracted white willow bark to evoked release of NO from pooled M. edulis pedal ganglia in Example 11.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION

In one embodiment, the invention provides a pharmaceutical composition including active chemical agents isolated from plant tissue and materials that stimulate the production of nitric oxide in pedal ganglia and human endothelial cells. Low molecular weight extracts from any of the plants listed below contain various amounts of the active chemical agents that stimulate production of NO. In other embodiments, the invention provides methods and materials for identifying and isolating additional active chemical agents having NO stimulating properties from other plants having such activity and methods and materials useful in the treatment of diseases and conditions requiring modification of cellular levels of NO.

Such active chemical agents contain at least two overlapping groups of compounds, the at least two groups of compounds known respectively as Healthin 1 and Healthin 2. Healthin 1 includes at least three compounds: 2,3-dihydroxypropyl oleate; bis(m-phenoxyphenyl)ether; and 6-acetyl-5,6,6a,7-tetrahydro-4H-dibezo(de,g)quinoline. Healthin 2 includes at least four compounds: 2,3-dihydroxypropyl oleate; bis(m-phenoxyphenyl)ether; 6-acetyl-5,6,6a,7-tetrahydro-4H-dibezo(de,g)quinoline; and (+)-N-(p-(2-methylbutoxy)benzylidene)-4-(2-methylbutyl)aniline.

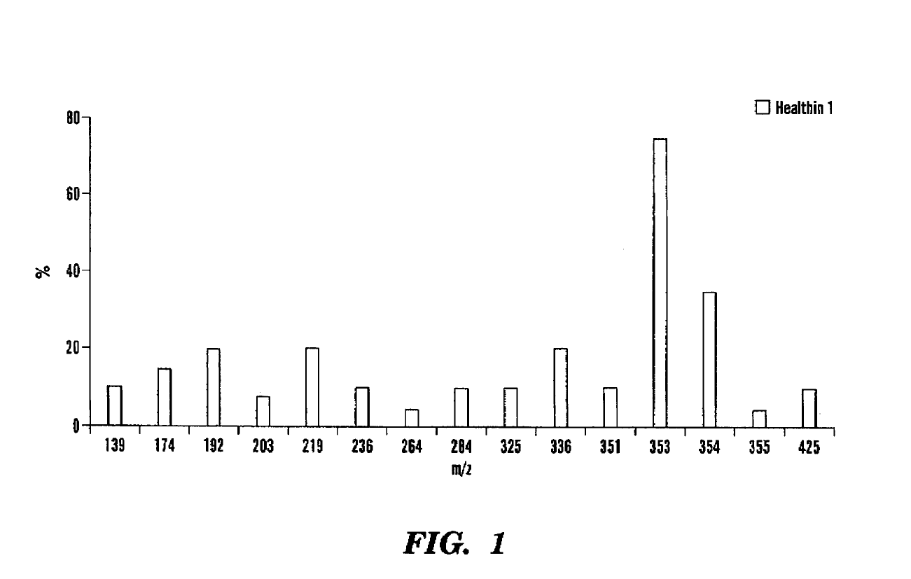

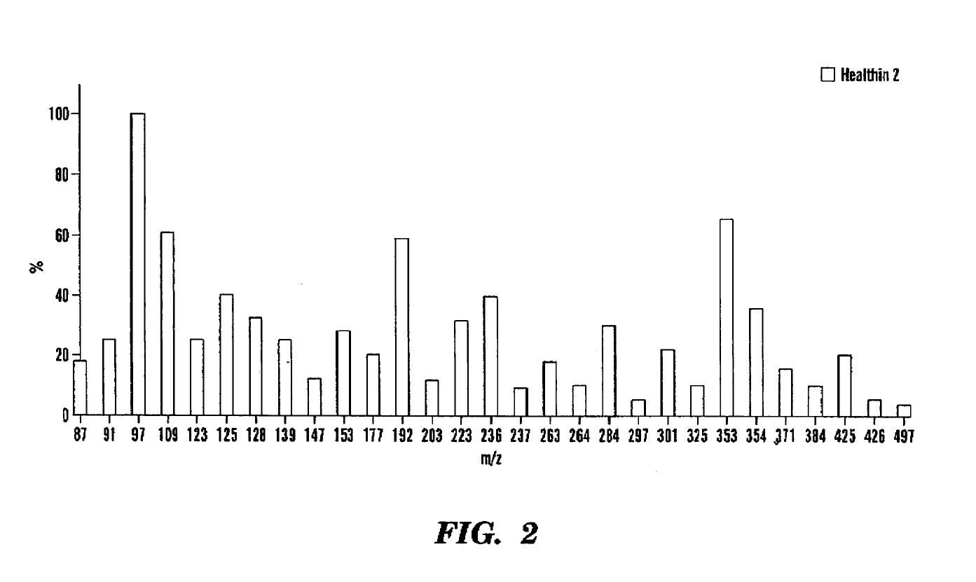

Referring to FIGS. 1 and 2, the mass spectrometry respectively for Healthin 1 and Healthin 2 in accordance with one embodiment of the invention is shown. Healthin 1 shows a major peak at 353 m/z. (FIG. 1) Healthin 2 shows major peaks at 97, 109, 192, and 353 m/z. (FIG. 2)

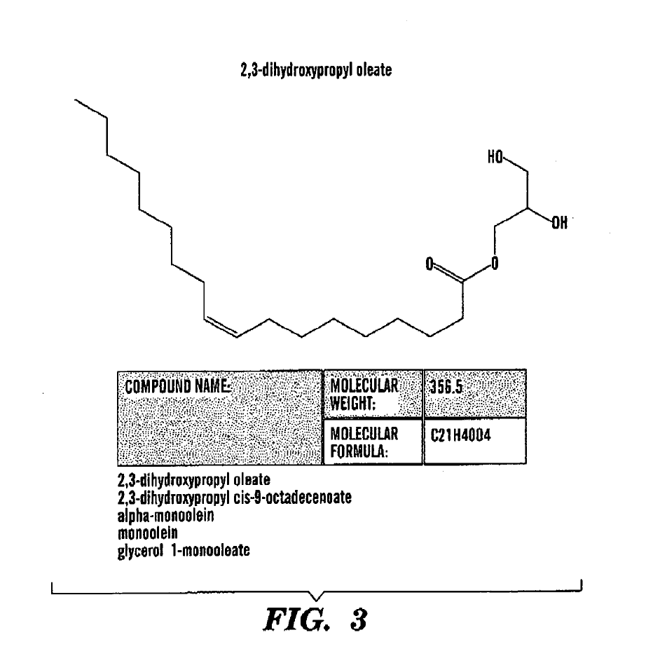

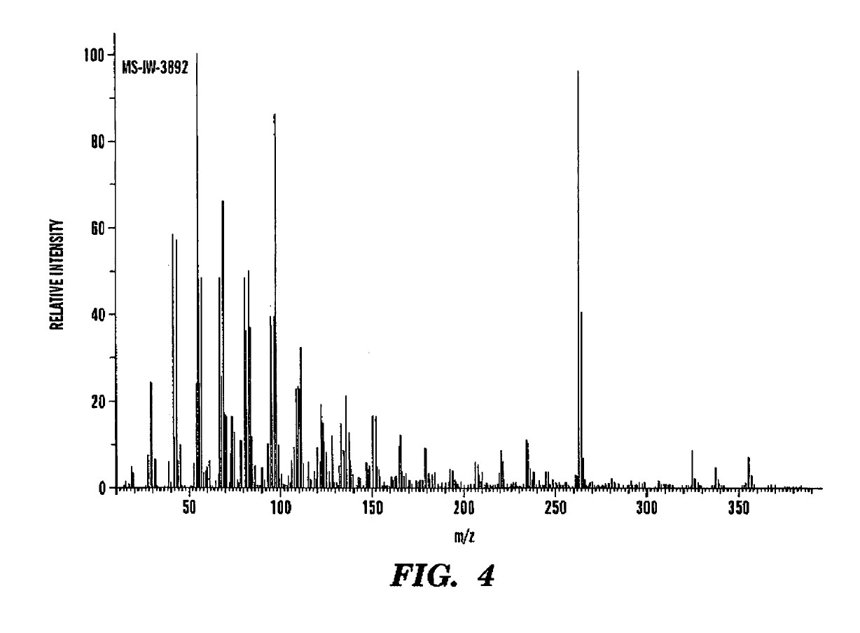

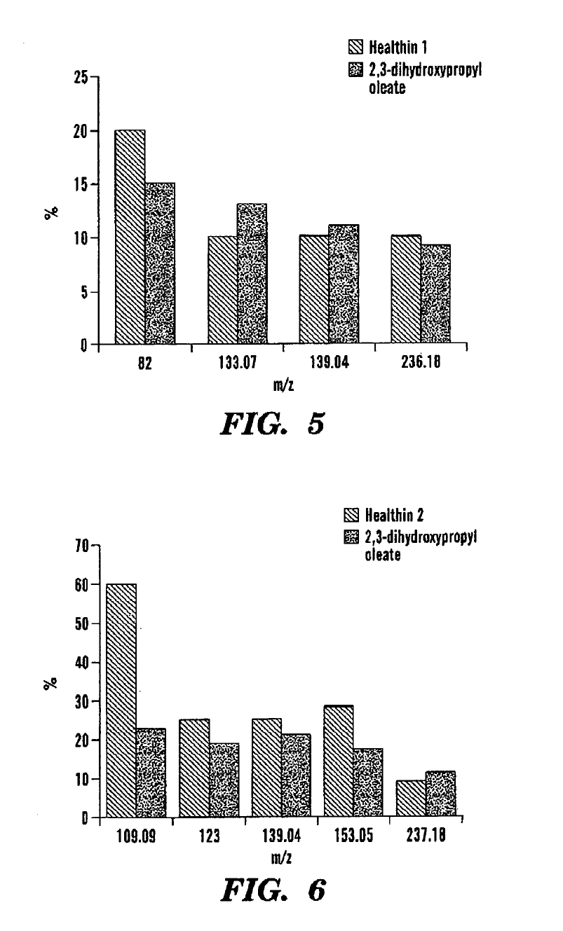

Referring to FIGS. 3-6, structure and mass spectrometry analysis for 2,3-dihydroxypropyl oleate is shown. FIG. 3 shows the structure of 2,3-dihydroxypropyl oleate. The molecular weight of 2,3-dihydroxypropyl oleate is approximately 356.5 daltons. The molecular formula is C₂₁H₄₀O₄. Alternative names for 2,3-dihydroxypropyl oleate include 2,3-dihydroxypropyl cis-9-octadecenoate; alpha-monoolein; monoolein; and glycerol 1-monooleate. FIG. 4 shows a mass spectrometry analysis for 2,3-dihydroxypropyl oleate. FIG. 5 shows a comparative mass spectrometry analysis illustrating a comparison between Healthin 1 and 2,3-dihydroxypropyl oleate. FIG. 6 shows a comparative mass spectrometry analysis illustrating a comparison between Healthin 2 and 2,3-dihydroxypropyl oleate.

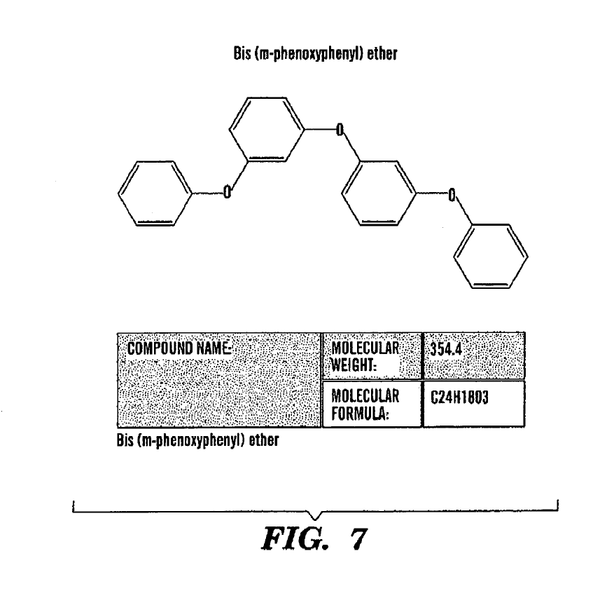

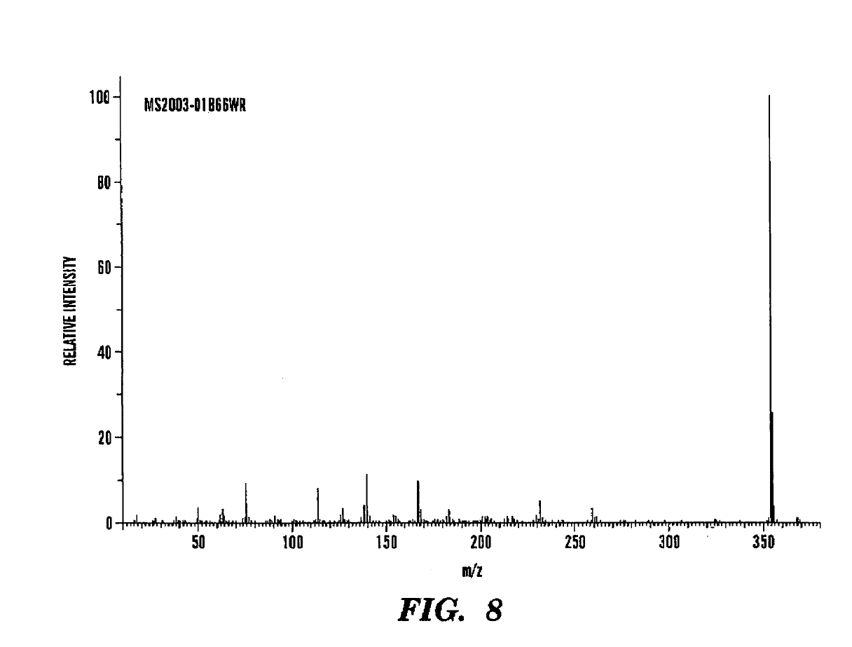

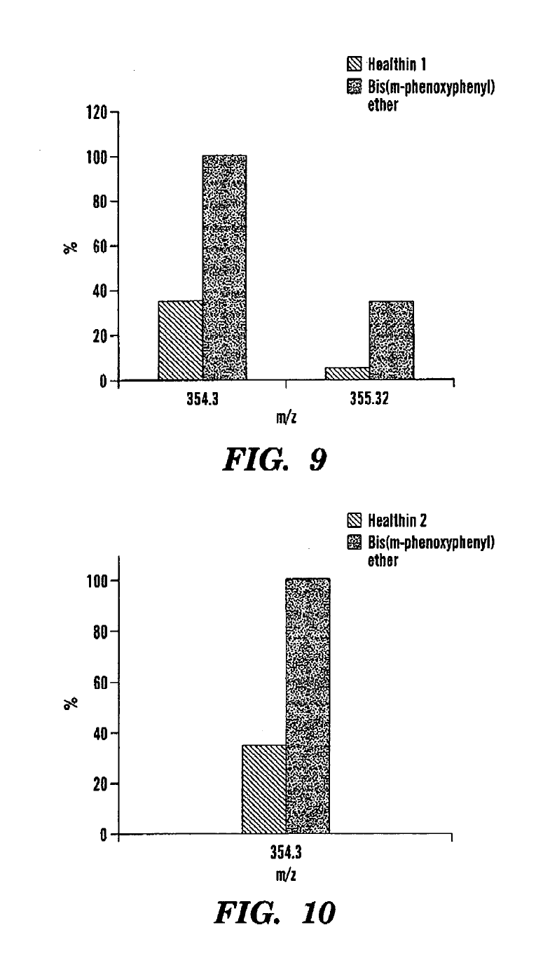

Referring to FIGS. 7-10, structure and mass spectrometry analysis for bis(m-phenoxyphenyl)ether is shown. FIG. 7 shows the structure of bis(m-phenoxyphenyl) ether. The molecular weight of bis(m-phenoxyphenyl)ether is approximately 354.4 daltons. The molecular formula is C₂₄H₁₈O₃. FIG. 8 shows a mass spectrometry analysis for bis(m-phenoxyphenyl)ether. FIG. 9 shows a comparative mass spectrometry analysis illustrating a comparison between Healthin 1 and bis(m-phenoxyphenyl)ether. FIG. 10 shows a comparative mass spectrometry analysis illustrating a comparison between Healthin 2 and bis(m-phenoxyphenyl)ether.

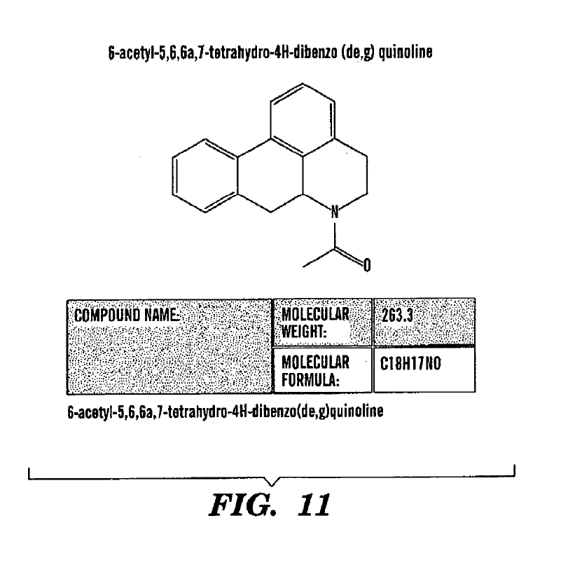

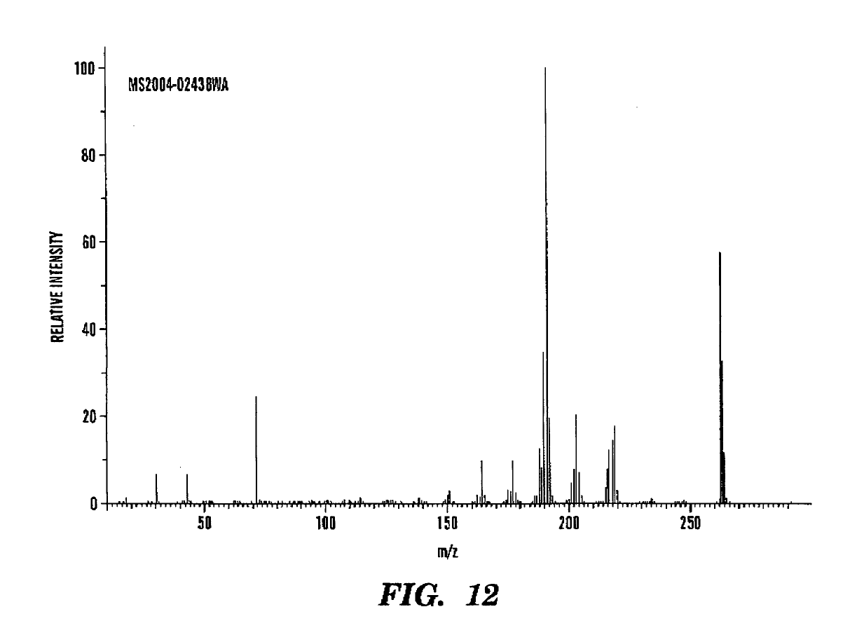

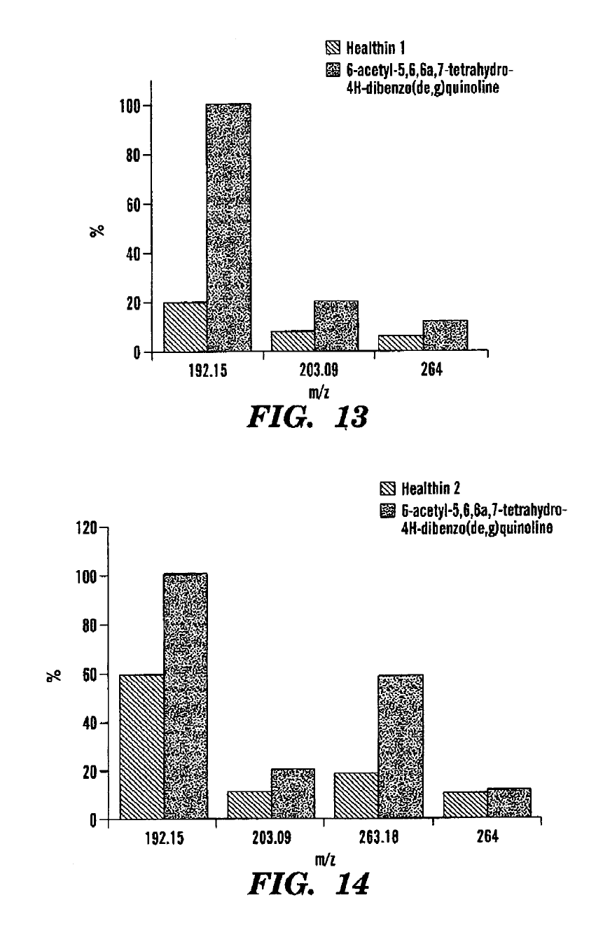

Referring to FIGS. 11-14, structure and mass spectrometry analysis for 6-acetyl-5,6,6a,7-tetrahydro-4H-dibezo(de,g)quinoline is shown. FIG. 11 shows the structure of 6-acetyl-5,6,6a,7-tetrahydro-4H-dibezo(de,g)quinoline. The molecular weight of 6-acetyl-5,6,6a,7-tetrahydro-4H-dibezo(de,g)quinoline is approximately 263.3 daltons. The molecular formula is C₁₈H₁₇NO. FIG. 12 shows a mass spectrometry analysis for 6-acetyl-5,6,6a,7-tetrahydro-4H-dibezo(de,g)quinoline. FIG. 13 shows a comparative mass spectrometry analysis illustrating a comparison between Healthin 1 and 6-acetyl-5,6,6a,7-tetrahydro-4H-dibezo(de,g)quinoline. FIG. 14 shows a comparative mass spectrometry analysis illustrating a comparison between Healthin 2 and 6-acetyl-5,6,6a,7-tetrahydro-4H-dibezo(de,g)quinoline.

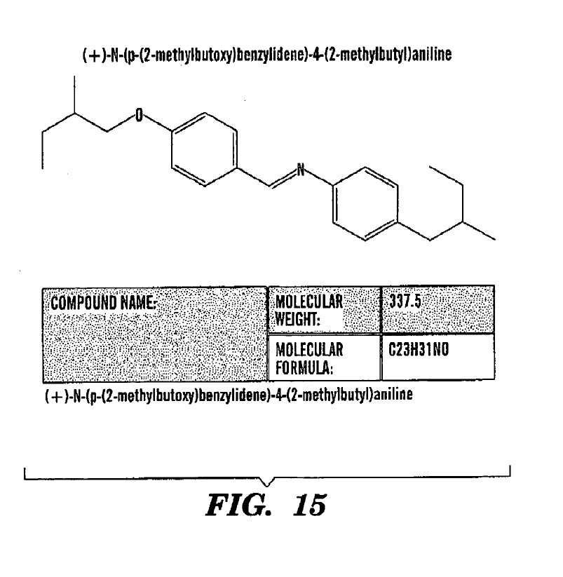

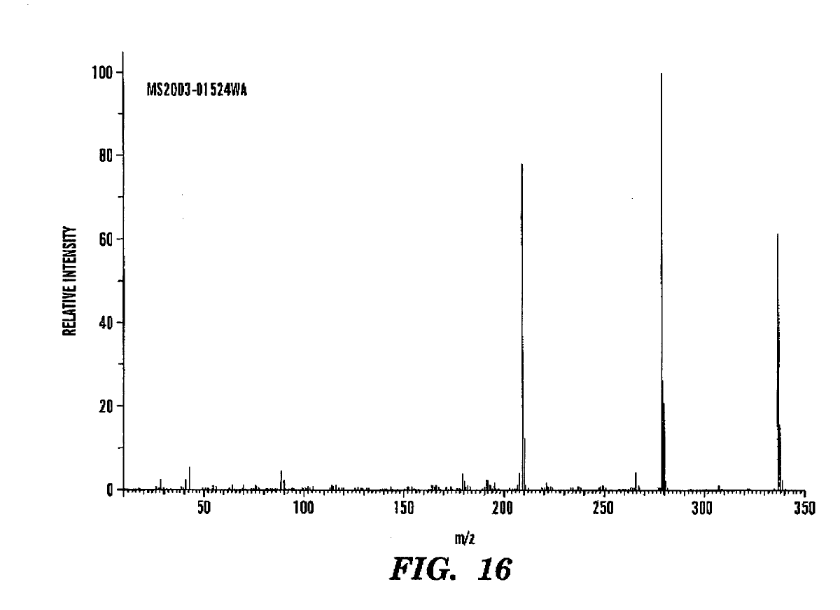

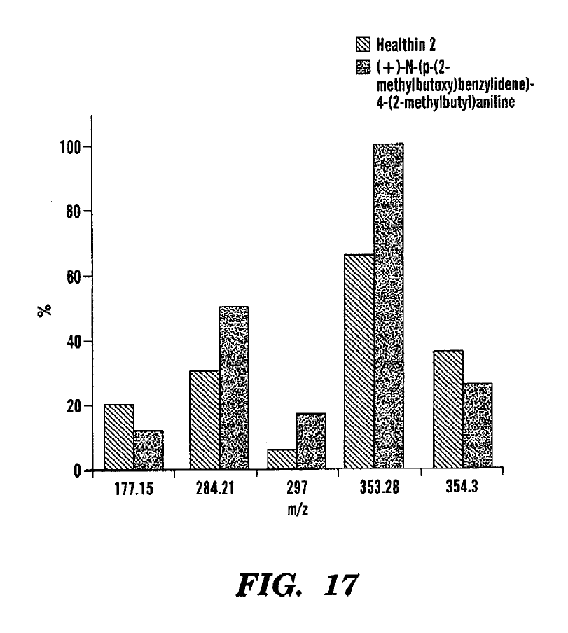

Referring to FIGS. 15-17, structure and mass spectrometry analysis for (+)-N-(p-(2-methylbutoxy)benzylidene)-4-(2-methylbutyl)aniline is shown. FIG. 15 shows the structure of (+)-N-(p-(2-methylbutoxy)benzylidene)-4-(2-methylbutyl)aniline. The molecular weight of (+)-N-(p-(2-methylbutoxy)benzylidene)-4-(2-methylbutyl)aniline is approximately 337.5 daltons. The molecular formula is C₂₃H₃₁NO. FIG. 16 shows a mass spectrometry analysis for (+)-N-(p-(2-methylbutoxy)benzylidene)-4-(2-methylbutyl)aniline. FIG. 17 shows a comparative mass spectrometry analysis illustrating a comparison between Healthin 2 and (+)-N-(p-(2-methylbutoxy)benzylidene)-4-(2-methylbutyl)aniline.

The active chemical agents, individually or in combination, are additionally characterized as having:
(i) the ability to stimulate nitric oxide release in the range of 15 nM to 100 nM in pedal ganglia cells;
(ii) the ability to stimulate nitric oxide release in the range of 50 nM to 100 nM in endothelial cells;
(iii) a single major peak on high performance liquid chromatographic analysis in 10 nM sodium chloride, 0.5 mM EDTA, 100 mM sodium acetate and 50% acetonitrile, pH 5.0; and/or
(iv) a retention time selected from a group consisting of: 15.8 minutes and 16.5 minutes.

The active chemical agents of the invention may be further characterized by being and having a molecular mass of between about 50 and about 5000 Daltons, or between about 50 and about 2500 Daltons, or between about 50 and about 1000 Daltons, or between about 50 and about 500 Daltons.

The extracts including the active chemical agents of the invention can be isolated from plants selected from the group consisting of Allium vineale, Salix alba, Agropyrum spp., Petroselinium crispum, Taraxacum officinale, Sesamum indicum, Medicago spp., Piper methysticum, Anthemis spp., Turnera diffusa, Verbascum densiflorum, Ocimum spp., Maranta arundinaceae, Coriandrum sativum, Artemesia dracunculus, Lavendula augustifolia, Mentha pulegium, Centella asiatica, Ginko biloba and Vitis vinifera.

One method of isolating and extracting to obtain the active chemical agents may comprise homogenizing dried plant material in an acidic solution followed by alcohol extraction and centrifugation for filtration to separate the solid material. The supernatant may be dried and then dissolved in an aqueous solution containing trifluoroacetic acid and subjected to solid phase extraction. The elute may be collected and further purified using high performance liquid chromatography. The extracted low molecular weight, active chemical agents may be further identified and characterized by mass spectrometric analysis.

These extracts including the active chemical agents are useful in the preparation of pharmaceutical compositions for treating antimicrobial infections such as bacterial infections and viral infections, and asthma, and/or other inflammatory conditions in mammals, especially in humans. The extracts, as detailed below, exhibit antibacterial, antinflammatory and anticancer effects. Consequently, pharmaceutical compositions comprising such extracts may be administered in the treatment various diseases and conditions in which antibacterial, antinflammatory or anticancer effects are desired, such as for example, in microbial infections. Alternatively, the pharmaceutical compositions of the invention may be employed as prophylactics. To form the extracts into pharmaceutical compositions, they may be dried, alone or in various combinations, and formed into pharmaceutical compositions comprising powders, tablets, poltices, pastes, creams, plasters, capsules and the like, with or without pharmaceutically acceptable excipients and/or adjuvants, in accordance with well known methods and techniques, for example, as detailed in Remington's Pharmaceutical Sciences, A. R. Gennaro, ed., Mack Publ. Co. Easton, Pa., 1985.

Pharmaceutical compositions useful in the practice of this invention include suitable dosage forms for oral, parenteral (including subcutaneous, intramuscular, intradermal and intravenous), transdermal, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. The pharmaceutical compositions may be prepared by conventional techniques appropriate to the desired preparation containing appropriate amounts of iloperidone or an active metabolite thereof. See, for example, Remington's Pharmaceutical Sciences, Mack Publishing Company, Easton, Pa., 17th edition, 1985.

In making pharmaceutical compositions for use in the invention, the active ingredient(s) will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the composition can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

The compositions are preferably formulated in a unit dosage form. The term “unit dosage form” refers to physically discrete units suitable as unitary dosages for human subjects and other mammals, each unit containing a predetermined quantity of active material calculated to produce the desired prophylactic or therapeutic effect over the course of a treatment period, in association with the required pharmaceutical carrier.

The invention will be further described in the following examples, without limiting the scope of the invention as described in the claims. In the examples, the plant extracts were made from the leaves of the plant, unless otherwise specified.

### EXAMPLES

### Example 1

### Extraction of Healthin 1 from Wheat Grass

Example 1 illustrates one method of extracting Healthin 1 from wheat grass. One gram of dried wheat grass plants, Agropyron spp. were homogenized in 1N HCl (0.5 g/ml). The resulting homogenates were extracted with 5 ml chloroform/isopropanol 9:1. After 5 min at room temperature, homogenates were centrifuged at 3000 rpm for 15 min. The supernatant was collected and dried with a Centrivap Console (Labconco, Kansas City, Mo.). The dried extract was then dissolved in 0.05% trifluoroacetic acid (TFA) water before solid phase extraction. Samples were loaded on a Sep-pak Plus C-18 cartridge (Waters, Milford, Mass.) previously activated with 100% acetonitrile and washed with 0.05% TFA-water. Morphine elution was performed with a 10% acetonitrile solution (water/acetonitrile/TFA, 89.5%:10%:0.05%, v/v/v). The eluted sample was dried with a Centrivap Console and dissolved in water prior to high performance liquid chromatography analysis (HPLC).

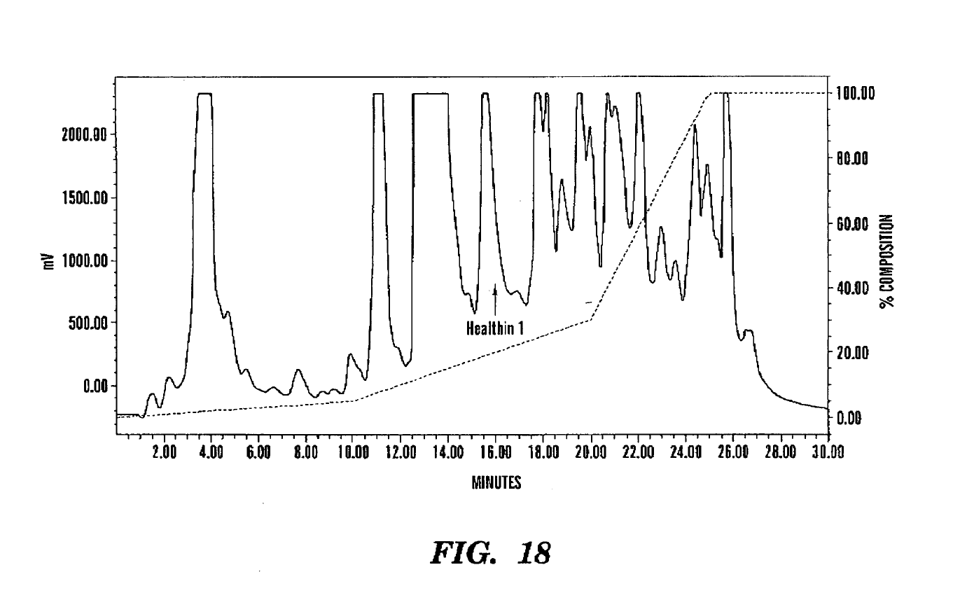

Reverse phase HPLC analysis using a gradient of acetonitrile was performed on a C-18 Unijet microbore column (BAS, West Lafayette, Ind.) using a Waters 626 pump (Waters, Milford, Mass.). 0.025 g dry weight of the wheat grass from the above-described extraction was used. The mobile phases were: Buffer A: 10 mM sodium chloride, 0.5 mM EDTA, 100 mM sodium acetate, pH 5.0; Buffer B: 10 mM sodium chloride, 0.5 mM EDTA, 100 mM sodium acetate, 50% acetonitrile, pH 5.0. A flow splitter (BAS), with split ratio 1/9 was used to provide the low volumetric flow rates required for the microbore column. Operating the pump at 0.5 ml/min yielded a microbore column flow rate of approximately 50 μl/min. The injection volume was 5 μl. The running conditions were: 0 min, 0% Buffer B; 10 min, 5% Buffer B; 25 min, 50% Buffer B; 30 min, 100% Buffer B. Both buffers were filtered through a Waters 0.22 μm filter and the temperature of the system was maintained at 25° C. The active agent (Healthin 1) extracted from the wheat grass had a retention time of 15.8 min (see arrow on FIG. 18). This result was repeated in 5 extractions. Several blank runs were performed between each of the 5 sample runs to prevent residual chromatography corresponding to the elution of the active component.

Active component detection was performed with an amperometric detector LC-4C (BAS). The microbore column was coupled directly to the detector cell to minimize the dead volume. The electrochemical detection system used a glassy carbon-working electrode (3 mm) and a 0.02 Hz filter (500 mV; range 10 nA). The cell volume was reduced by a 16 μm gasket. The chromatographic system was controlled by the Waters Millennium chromatography Manager V3.2 software and the chromatograms were integrated with Chromatograph software (Waters). The concentration was extrapolated from the peak area. The average concentration in the 5 samples was 1 μg/gm dry weight. Blank runs between determinations failed to elicit carry over residue. The fractions from each of the 5 runs were collected, dried and applied in the NO tissue assays described below. Results are illustrated in FIG. 18.

An alternative method of purification was performed by methanol extraction followed by HPLC purification on a Spherisorb column as follows. One gram of wheat grass, Agropyron spp, was homogenized in 50% methanol, 50% purified water, extracted with 50% methanol, and dried by speed vacuum. The sample was stored at −20° C. HPLC purification was carried out with a two solvent system: Buffer A was composed of 10 mM 1-heptane sulfonic acid, sodium salt and 10 mM sodium phosphate monobasic water, pH 3; Buffer B was composed of 10 mM 1-heptane sulfonic acid, sodium salt and 10 mM sodium phosphate monobasic, 50% methanol. The injection volume was 10 microliters. The running conditions were: 0-10 min, 50% Buffer B; 10-20 min, Buffer B increased from 50 to 100%; 25 min, 100% Buffer B; 35 min, 50% Buffer B. Fractions were collected from 0 to 30 minutes after sample injection. The collected fractions were dried by speed vacuum and maintained at −20° C. The active agent extracted from the wheat grass had a retention time of 16 min (see arrow on FIG. 18).

### Example 2

### Extraction of Healthin 2 from White Willow Bark

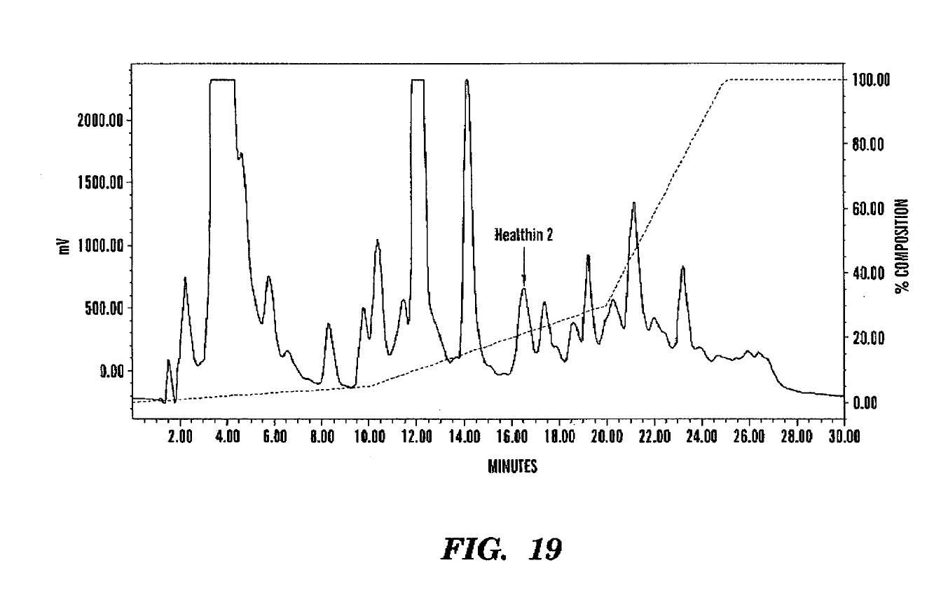

Example 2 illustrates one method of extracting Healthin 2 from white willow bark. The identical procedure was performed with 0.02 grams (dry weight) of white willow bark, Salix alba, The active agent (Healthin 2) extracted from the white willow bark had a retention time of 16.50 min. The average concentration in the 5 samples sun was 0.3 μg/gm dry weight. See FIG. 19.

### Example 3

### Mass Spectrometric Identification of Active Chemical Agents from Wheat Grass

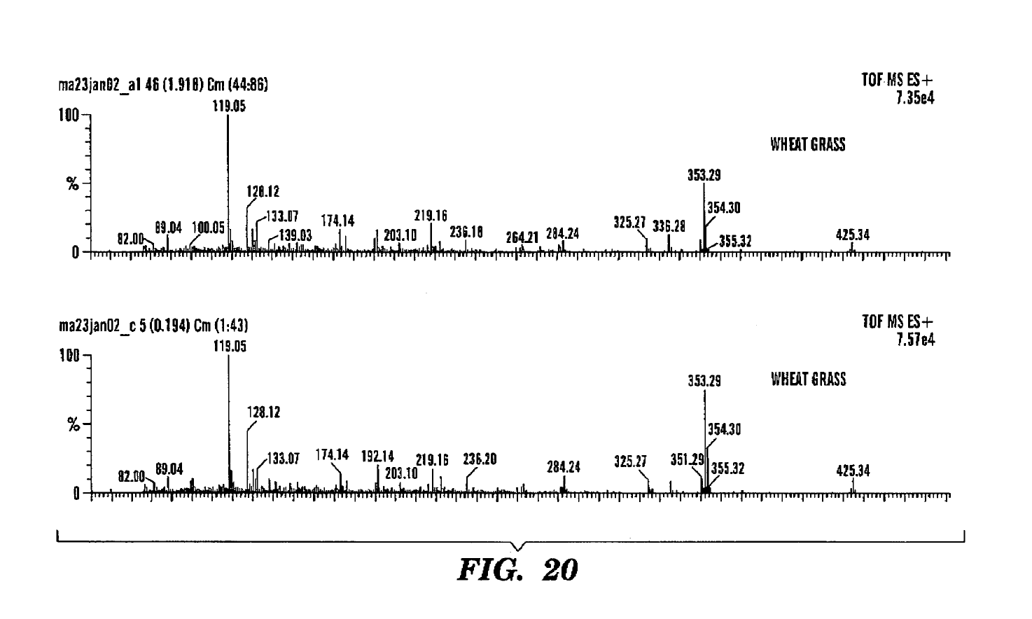

Example 3 illustrates mass spectrometric identification of the active chemical agents from wheat grass. The HPLC fraction, 1/100 microliters, containing the NO releasing activity from the first purification detailed in Example 1 above was subjected to nano electrospray ionization double quadrupole orthogonal acceleration Time of Flight mass spectrometry (Q-TOF-MS) on a Micromass Q-TOF system (Micromass, UK) as follows. One μl of acetonitril/water/formic acid (50:49:1, v/v/v) containing the sample was loaded in a gold-coated capillary Micromass F-type needle. The sample was sprayed at a flow rate of 30 nl/min, giving an extended analysis time during which MS spectrum and several MS/MS spectra were acquired. During MS/MS, or tandem mass spectrometry, fragmentations are generated from a selected precursor ion by collision-induced dissociation (CID). Since not all ions fragment with the same efficiency, the collision energy is typically varied between 20 and 35 V, so that the parent ion is fragmented into a satisfying number of different daughter ions. Needle voltage was set at 950 and cone voltage was set at 25. The instrument was operated in the positive mode. The results are illustrated in FIG. 20. Healthin 1, the active agent isolated and purified from the wheat grass sample, yielded major signals at 353.28 and 119.05 daltons.

### Example 4

### Mass Spectrometric Identification of Active Agents from Salix alba

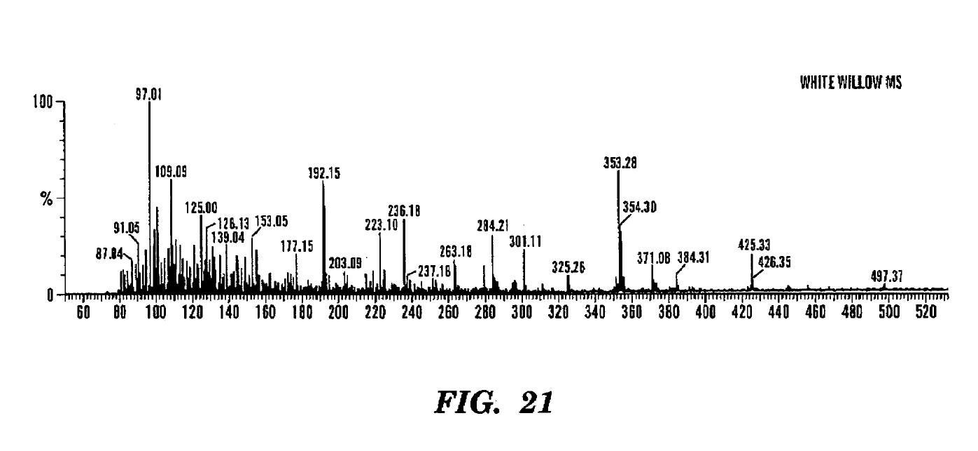

Example 4 illustrates mass spectrometric identification of the active chemical agents from white willow bark. The identical procedure from Example 3 was performed with one gram of white willow bark, Salix alba. The results are shown in FIG. 21. Healthin 2, the active agent isolated and purified from white willow bark sample, yielded major signals at 353.28, 192.15, 109.09 and 97.1 daltons.

### Example 5

### Wheat Grass Extract Stimulation of NO in Pedal Ganglia and Endothelial Cells

Example 5 illustrates wheat grass extract stimulation of NO release in pedal ganglia and endothelial cells. Ten Mytilus edulis pedal ganglia, dissected from live animals, were placed in 1.5 ml Eppendorf tubes with 990 μl of phosphate buffer saline (PBS). Cultured human vein endothelial cells (ATCC # CRL 1730) were washed in PBS at 4° C. The vein endothelial cells were grouped into patches of approximately 10⁶ cells each and placed in 990 μl of PBS at 4° C. One gram of dried wheat grass, Agropyron spp, was purified by HPLC as detailed above and the fraction corresponding to the retention time of the Healthin 1 was collected and dried. The fraction was then reconstituted in 20 μl PBS. 10 μl were added to the tubes containing the ganglia or the endothelial cells or PBS alone (control). NO production was determined using a Mark II isolated nitric oxide meter (World Precision Instruments, Sarasota, Fla.) fitted with a 200 μM sensor. If a response was detected in the tube containing PBS alone, the amount was subtracted from the amounts detected in the tubes containing the tissue samples.

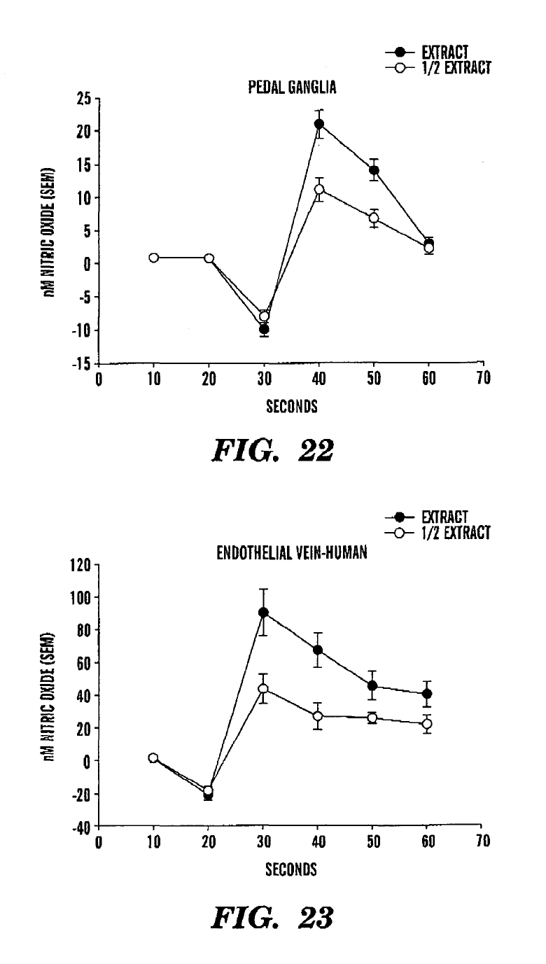

The results are shown in FIGS. 22 and 23. The pedal ganglia tube cells released 17 nM NO (FIG. 22), the human endothelial cells released 91 nM NO (FIG. 23). The identical volume added to the control tube resulted in the production of <3 nM NO.

### Example 6

### White Willow Bark Extract Stimulation of NO in Pedal Ganglia and Endothelial Cells

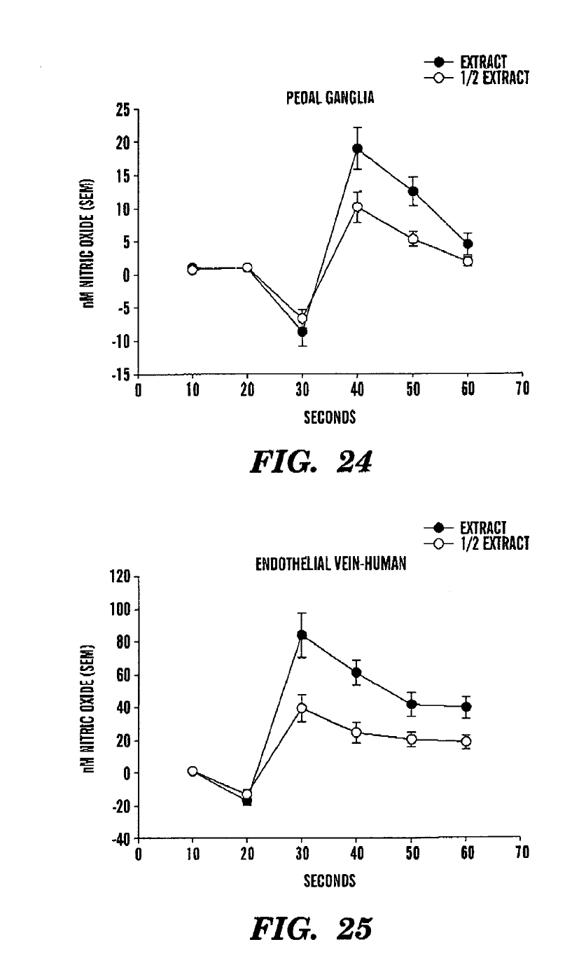

Example 6 illustrates white willow bark extract stimulation of NO release in pedal ganglia and endothelial cells. The procedure detailed in Example 5 above was performed with one milligram of the agent purified from the white willow bark, Salix alba, from Example 2. The results are shown in FIGS. 24 and 25. The pedal ganglia tube cells released 19 nM NO (FIG. 24), the human endothelial cells released 87 nM NO (FIG. 25). The identical volume added to the control tube resulted in the production of <3 nM NO.

### Example 7

### Analysis of Plants of Various Species for NO Release

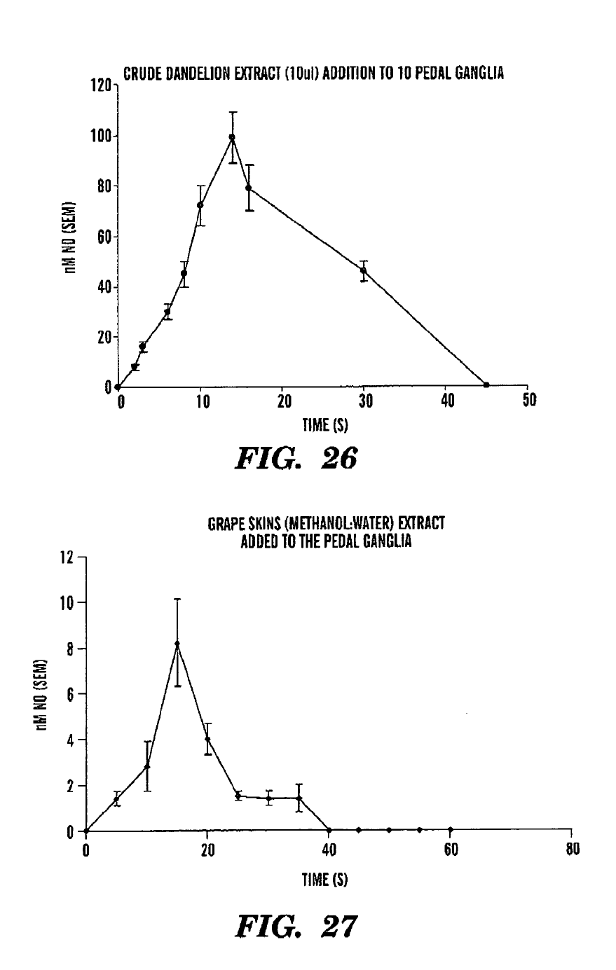

Example 7 illustrates an analysis of extracts of plants for NO release properties. Employing the isolation and purification techniques described above, a variety of herbaceous plants were analyzed for their ability to release cNOS—derived nitric oxide in the pedal ganglia and in publicly available SK-N-MC (ATCC # HBT-10) and PC-12 (ATCC # CRL 1721) cells. These results are set forth in Tables I, II, and III below. In Table I, a plus sign indicates detection of at least 1 nM nitric oxide. A minus sign indicates no detection or detection of less than 1 nM nitric oxide. In Table II, results in the SK-N-MC cell line are set forth; the concentration of plant material used and the quantity of NO detected is indicated. In Table II, the designation Reactive in PBS indicates that the extract spontaneously released NO into PBS buffer in the absence of biological tissue. In Table III, results are set forth for the identical procedures performed using the ganglia cell line. The types of plant materials employed are indicated, for example flowers, leaves, roots, rhizomes, stems, bark. Where not specified, leaves were employed. FIG. 26 shows an exemplary result.

TABLE I
NO determination of ganglia, SK-N-MC and PC-12 cells
treated with various plant extractions. Blank indicates
plant not tested in that cell line.

| | | SK- | PC- |
|---|---|---|---|
| | Ganglia | N-MC | 12 |
| Allium vineale (Garlic) | −−− | + | |
| Salix alba (White willow) bark | + | + | |
| Agropyron (Wheat grass) | + | + | |
| Petroselinium crispum or Carum petroselinum | −−− | + | |
| (Parsley) | | | |
| Taraxacum officinale (Dandelion) | + | −−− | |
| Sesamum indicum (Sesame, Gin sum) leaves | | + | |
| Medicago spp. (Alfalfa) | | + | |
| Piper methysticum (Kava) | | + | |
| Anthemis spp. (Chamomile) | | +++ | + |
| Turnera diffusa (Damian) | | | + |
| Verbascum densiflorum (Mullein) | | | + |
| Maranta arundinaceae (Arrowroot) roots | | | −−− |
| Lavandula angustifolia (Lavender) flower | | | −−− |
| Ocimum spp. (Sweet basil) | | | −−− |
| Artemesia dracunculus (Tarragon) leaves | | | −−− |
| Aloe vulgaris or A. barbadensis (Aloe) leaves | −−− | −−− | |
| Vacciuium membranaceum (Bilberry) | −−− | −−− | |
| Brassica spp. (Cabbage) | −−− | −−− | |
| Daucus carota (Carrot) | −−− | −−− | |
| Zea mays flowers (corn silk) | −−− | −−− | |
| Echinacea (Coneflower) | −−− | −−− | |
| Lactuca spp. (Lettuce) | −−− | −−− | |
| Tabebuia impetiginosa, T. avellanedai, | −−− | −−− | |
| Tecoma curialis (Pau d'arco) | | | |
| Mentha piperita (Peppermint) | −−− | −−− | |
| Rubus spp. (Raspberry) | −−− | −−− | |
| Rosmarinus officinalis (Rosemary) | −−− | −−− | |
| Salvia spp. (Sage) | −−− | −−− | |
| Equisetum hyemale (Shave grass) | −−− | −−− | |
| Ulmus rubra, Fremontodendron californicum | −−− | −−− | |
| (Slippery elm) bark | | | |
| Phaseolus spp. (String bean) | −−− | −−− | |
| Thymus spp. (Thyme) | −−− | −−− | |

TABLE II
NO determination of SK-N-MC cells treated
with various plant extractions

| | | Results |
|---|---|---|
| | Concentration | (nM) |
| Ocimum spp. (Basil) | 6 mg of crude extraction | 31 |
| Verbascum densiflorum (Mullein) | 6 mg of crude extraction | No effect |
| Turnera diffusa (Damian) | 6 mg of crude extraction | No effect |
| Maranta arundinaceae | 6 mg of crude extraction | 31 |
| (Arrowroot) root | | |
| Coriandrum sativum (Cilantro) | 6 mg of crude extraction | 172 |
| Artemesia dracunculus (Tarragon) | 6 mg of crude extraction | 135 |
| Lavendula augustifolia (Lavender) | 6 mg of crude extraction | 48 |
| flower | | |
| Mentha pulegium (Pennyroyal) | 6 mg of crude extraction | 66 |
| Quercetine* | 6 mg of crude extraction | 14 |
| Piper methysticum (Kava) | 1.5 mg | 108 |
| Anthemis spp. (Chamomile) | 1.5 mg | 31 |
| Centella asiatica (Gotu kola) | 1.5 mg | Reactive |
| | | in PBS |
| Scutellaria lateriflora (Skullcap) | 1.5 mg | Negative |
| Ginko biloba (Ginko) | 1.5 mg | Reactive |
| | | in PBS |
| Hypericum perforatum (St John's | 1.5 mg | Negative |
| Wort) | | |
| Urtica dioeca (Common nettle) | 1.5 mg | Negative |

*Quercetine (from Sigma Chemicals) is a plant flavanoid found in many plants, and especially in fruits.

TABLE III
NO determination of ganglia cells treated
with various plant extractions

| | | |
|---|---|---|
| Anthemis spp. (Chamomile) | 6 mg of crude extraction | 67 nM |
| Piper methysticum (Kava) root | 6 mg of crude extraction | 13 nM |
| Turnera diffusa (Damian) | 6 mg of crude extraction | 22 nM |
| Verbascum densiflorum (Mullein) | 6 mg of crude extraction | 15 nM |
| Ocimum spp. (Basil) | 6 mg of crude extraction | 19 nM |

### Example 8

### Grape Skin Extraction and NO release

Example 8 illustrates grape skin extract stimulation of NO release in pedal ganglia. Ten grams (wet weight) of black grape skins, Vitis vinifera, were placed in a 50 ml Falcon tube with 15 ml of a 1:1 mixture of methanol or ethanol and water. The tubes were shaken overnight at room temperature and the resulting extracts were aliquoted, 1 ml per tube, into twelve 1.5 ml Eppendorf tubes. The tubes were evaporated to dryness in a speedvac and then reconstituted in 1 ml phosphate buffered saline (PBS) solution. 10 μg o this solution was used to treat the invertebrate nervous tissue pedal ganglia (see Example 5, above) and NO release was measured in real time by an amperometric probe specific for the measurement of NO. Grape skin extracted in methanol caused a release of NO within 15 seconds of treatment (see FIG. 27) whereas grape skin extracted in ethanol did not (within the same time period). NO release was not observed when the extract (either methanol or ethanol extracted) was added to PBS alone.

### Example 9

### Anti-Microbial Effects of Extracts on Cells

Example 9 illustrates the anti-microbial effects of extracts including the active chemical agents on cells. A dried, powdered, formulation of a 1:1 mixture of the wheat grass extract and white willow bark extract prepared in Example 1 above was tested for its ability to inhibit bacterial growth in culture. The formulation was reconstituted in 10 ml of LB broth (Amersham Biosciences, Inc.). The broth was then inoculated with E. coli bacteria and incubated for 5 and 24 hours at 37° C. 20 μl of the cultures were streaked on LB-agar plates and incubated overnight at 37° C. There was no growth observed in the 5 and 24 hours bacterial cultures as compared to the control (LB broth alone).

An additional control experiment was conducted with the known antibacterial agent, SNAP. One μg/ml SNAP was added to LB broth. The broth was then inoculated with E. coli bacteria and incubated for 5 and 24 hours at 37° C. 20 μl of the cultures were streaked on LB-agar plates and incubated overnight at 37° C. Bacterial growth was decreased in the SNAP culture at 5 and 24 hours, as compared to the control.

This experiment demonstrates that the wheat grass/white willow extract of the invention exhibits greater antibacterial activity than the known antibacterial agent SNAP.

### Example 10

### Anti-Cancer Effects of Extracts on SK-N-MC Cells

Example 10 illustrates the anti-cancer effects of extracts including the active chemical agents on SK-N-MC cells. SK-N-MC cells were incubated with either garlic (Allium vineale) or parsley (Petroselinium crispum) extractions, 0.005 g/ml in RPMI media, for two days. The cells were then stained with Trypan Blue indicator (Invitrogen Corp.) and observed under a research microscope at 200×. Healthy cells do not allow this indicator to enter the cell wall whereas cells which turn blue are dead or dying because the reagent has entered the cytoplasm. Microscope observation of both garlic and parsley treated cells indicated almost 100% of the cells were dead. Similar results were observed with 1 N solutions of Mullein (Verbascum densiflorum), Kava (Piper methysticum), Chamomile (Anthemis spp.), and Damian (Turnera diffusa). Other plant extracts prepared and tested in a similar manner that induced cell death in SK-N-MC cells were Bilberry (Vaccinium myrtillus), Enchinaceae purpurae, Garlic (Allium vineale), Goldenseal (Hydrastis candensis), Parsley (Petroselenium crispum or C. petroselenium), Paul d'arco bark (Tabebuia impetiginosa), Rosemary (Rosmarinus officinalis), Slippery elm (Ulmus rubra or Fremontodendron californicum), and White willow bark (Salix alba). The strongest anti-cancer effects were seen with garlic and parsley.

Plant extracts prepared and tested in the same manner that exhibited no anti-cancer effect on SK-N-MC cells included Raspberry (Rubus spp.), Peppermint (Mentha piperita), Shave grass (Equisetum hyemale), cornsilk (Zea mays flowers), Dandelion (Taraxacum officinale), Alfalfa (Medicago spp.), Thyme (Thymus spp.) and Slippery Elm (Ulmus rubra and Fremontodendron californicum).

### Example 11

### NO Releasing Properties of an Extract of White Willow Bark

Traditional aqueous extractions of white willow bark have yielded herbal medicinal preparations with significant anti-pyretic, anti-inflammatory, and analgesic properties. The medicinal/therapeutic properties of white willow bark extracts have been attributed to water soluble molecules classified as non-steroidal anti-inflammatory drugs (NSAIDs). Prominent white willow bark NSAIDs include salicin [2-(Hydroxymethyl)phenyl β-D-glucopyranoside] and salicylic acid [2-hydroxybenzoic acid]. Historically, the prototype NSAID aspirin [acetylsalicylic acid; 2-acetyloxybenzoic acid] was synthesized via chemical acetylation of salicylic acid obtained from willow bark.

Specific HPLC fractions of white willow bark extracts have been demonstrated to evoke release of the therapeutically beneficial free radical gas nitric oxide (NO) from ex vivo tissue preparations. The temporal profile of NO release indicates selective stimulation of constitutive NO Synthase (cNOS), the NOS isozyme responsible for normal health-related vascular and organ function. QTOF mass spectroscopic analysis of active NO-releasing HPLC fractions indicate a lack of chemical identity with previously characterized salicin and salicylate analogs found in white willow bark. These data strongly support the existence of a novel class of non-salicin/salicylate therapeutic chemicals in white willow bark that displays an independent mode of action from that established for the pharmaceutical class of salicin/salicylate NSAID agents.

To provide additional confirmatory biochemical evidence that white willow bark contains novel class of non-salicin/salicylate anti-inflammatory compounds, we employed a traditional lipid extraction to selectively eliminate water soluble salicin/salicylate-related chemical compounds. Additionally, parallel water extractions were performed according to specifications listed in two prior art documents. Aliquots from lipid and water extracted white willow bark were tested for biological activity via evoked release of NO from nervous tissue.

White Willow Bark Extraction of Lipid Soluble Compounds: White willow bark was extracted according to a standard lipid purification protocol. A 10% extraction preparation employed 2 g of pulverized white willow bark incubated in 20 ml of organic solvent consisting of chloroform/2-propanol (ratio of 9:1) for 8 hrs at 4o. Supernatant fractions were collected by centrifugation and vacuum dried utilizing a Centri-Vap apparatus. Dried extraction residues were resuspended by sonication in cold PBS (phosphate buffered saline, pH 7.4) and clarified by centrifugation. Aliquots of clarified white willow bark lipid extracts were tested for their ability to release NO from ex vivo tissue preparations (below).

White Willow Bark Water Extraction: To demonstrate that NO releasing constituents of white willow bark are exclusively associated with lipid soluble fractions, a traditional water extraction was performed. Two known water extraction procedures were employed: 1) a 10% extraction of 2 g of pulverized white willow bark incubated in 20 ml dH2O for 8 hrs at room temperature, ref a. below; 2) a 10% extraction of 2 g of pulverized white willow bark incubated in 20 ml of boiling dH2O followed by natural cooling at room temperature, ref b. below. Extractions were clarified by centrifugation and supernatants were reserved and freeze dried. Dried samples were reconstituted in PBS and aliquots were tested for their ability to release NO from ex vivo tissue preparations.

Real-time Nitric Oxide Release Assay: Nitric oxide releasing activities of aliquots of clarified white willow bark lipid extracts were determined using a standardized ex vivo invertebrate neural tissue preparation in use in the laboratory for over ten years. For each independent analysis, 10 Mytilus edulis pedal ganglia (1-1.2 mg, wet weight/ganglia) were dissected on ice and placed in a 1.7-ml low-binding, pre-siliconized, microcentrifuge tube containing 1 ml of PBS. Nitric oxide release was directly measured using a NO-specific amperometric probe (30 μm, 0.5 mm, World Precision Instruments, Sarasota, Fla.). The amperometric probe was allowed to equilibrate for 10 minutes in the incubation medium (tissue-free) before being transferred to the tube containing the tissue, and allowed to equilibrate for another 5 minutes. A micromanipulator (World Precision Instruments, Sarasota, Fl), which is attached to the stage of an inverted microscope (Nikon Diaphot, Melville, N.Y.), was used to position the amperometric probe 15 μm above the tissue. NO released from each nervous tissue preparation was quantified using an Apollo 4000 Free Radical Analyzer with an NO-selective amperometric nanoprobe and proprietary software. A linear standard function was constructed from the measured amperiometric responses provided by predetermined concentrations of the NO donor S-nitroso-N-acetyl-DL-penicillamine (SNAP) in the presence of 0.1M CuCl2.

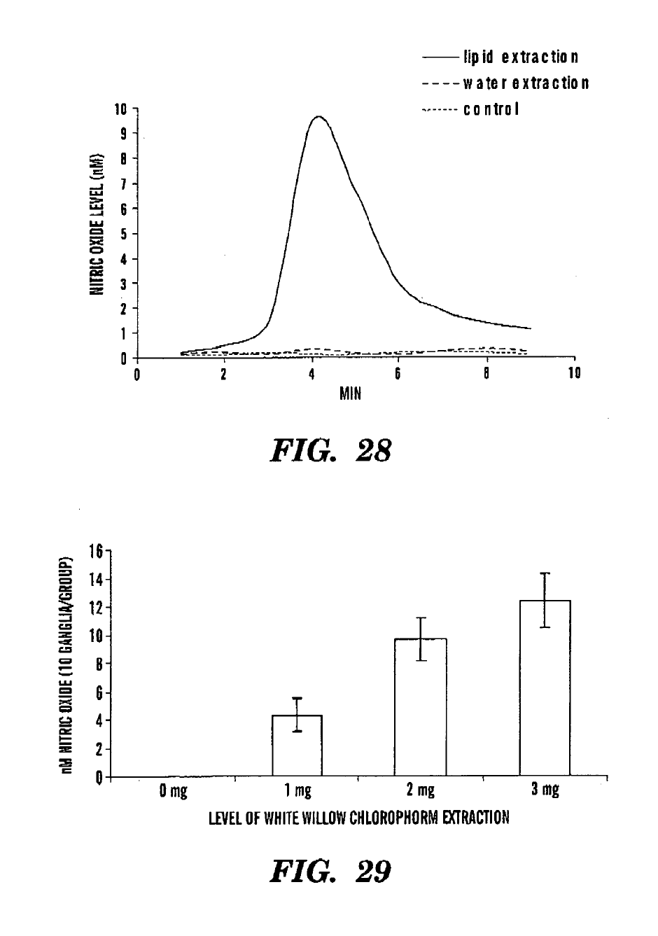

Results: FIG. 28 illustrates real-time evoked release of NO from pooled M. edulis pedal ganglia by a white willow bark lipid extract in comparison to cold and boiling water white willow bark water extracts. A 20 ul aliquot equivalent to 2 mg of lipid extracted white willow bark engendered release of NO into the tissue bath at a peak concentration of approximately 10 nM equivalent to 1 nM/ganglia (FIG. 28—upper continuous trace). In marked contrast to the lipid extraction protocol, 20 ul aliquots equivalent to 2 mg of cold and boiling water extracted white willow bark were observed to be without effect on evoked release of NO from pooled ganglia (FIG. 28-lower broken traces).

Aliquots of a reconstituted white willow lipid extract evoked the release of NO from pooled Mytilus edulis pedal ganglia in a concentration dependent manner. Typically, a 20 ul aliquot equivalent to 2 mg of extracted white willow bark engendered release of NO into the tissue bath at a peak concentration of 10 nM equivalent to 1 nM/ganglia (FIG. 28—upper solid trace). In marked contrast to the lipid extraction protocol, a 20 ul aliquots equivalent to 2 mg of both cold and boiling water extracted white willow bark were observed to be without effect on evoked release of NO from pooled ganglia (FIG. 28—lower broken traces).

FIG. 29 illustrates a dose response relationship of lipid extracted white willow bark to evoked release of NO from pooled M. edulis pedal ganglia. 10, 20, and 30 ul aliquots equivalent to 1, 2, and 3 mg equivalents of lipid extracted white willow bark engendered release of NO into the tissue bath at a peak concentrations of 4, 10, and 12 nM, respectively. Similar results were observed for 3 independent experiments utilizing pooled pedal ganglia.

Aliquots of both cold and boiling water extracted white willow bark equivalent to 1, 2, 5, and 10 mg of white willow bark (replicated 3 times) were observed to be without effect on evoked release of NO from pooled ganglia and produced similar time dependent negative responses. (FIG. 28—lower broken traces). Finally, control experiments demonstrated that equivalent aliquots of lipid extractable white willow bark added to PBS alone in the absence of pedal ganglia did not produce amperometric responses indicative of non-specific activation of the measurement electrode (not shown).

What is claimed is:

1. A pharmaceutical composition for stimulating nitric oxide production in mammalian cells, the pharmaceutical composition comprising:
at least one compound selected from a group consisting of: 2,3-dihydroxypropyl oleate; bis(m-phenoxyphenyl)ether; 6-acetyl-5,6,6a,7-tetrahydro-4H-dibezo(de,g)quinoline; and (+)-N-(p-(2-methylbutoxy)benzylidene)-4-(2-methylbutyl)aniline.

2. The pharmaceutical composition of claim 1, wherein the at least one compound includes 2,3-dihydroxypropyl oleate.

3. The pharmaceutical composition of claim 1, wherein the at least one compound includes bis(m-phenoxyphenyl)ether.

4. The pharmaceutical composition of claim 1, wherein the at least one compound includes 6-acetyl-5,6,6a,7-tetrahydro-4H-dibezo(de,g)quinoline.

5. The pharmaceutical composition of claim 1, wherein the at least one compound includes (+)-N-(p-(2-methylbutoxy)benzylidene)-4-(2-methylbutyl)aniline.

6. The pharmaceutical composition of claim 1, wherein the at least one compound is derived/extracted from at least one plant species selected from the group consisting of Allium vineale, Salix alba, Agropyrum spp., Petroselinium crispum, Taraxacum officinale, Sesamum indicum, Medicago spp., Piper methysticum, Anthemis spp., Turnera diffusa, Verbascum densiflorum, Ocimum spp., Maranta arundinaceae, Coriandrum sativum, Artemesia dracunculus, Lavendula augustifolia, Mentha pulegium, Centella asiatica, Ginko biloba and Vitis vinifera.

7. The pharmaceutical composition of claim 1, wherein the pharmaceutical composition has the ability to stimulate nitric oxide release in the range of 15 nM to 100 nM in pedal ganglia cells.

8. The pharmaceutical composition of claim 1, wherein the pharmaceutical composition has the ability to stimulate nitric oxide release in the range of 50 nM to 100 nM in endothelial cells.

9. A method of stimulating nitric oxide production in an individual in need of such treatment, the method comprising:
administering to the individual at least one compound selected from a group consisting of: 2,3-dihydroxypropyl oleate; bis(m-phenoxyphenyl)ether; 6-acetyl-5,6,6a,7-tetrahydro-4H-dibezo(de,g)quinoline; and (+)-N-(p-(2-methylbutoxy)benzylidene)-4-(2-methylbutyl)aniline.

10. The method of claim 9, wherein administering includes administering 2,3-dihydroxypropyl oleate.

11. The method of claim 9, wherein administering includes administering bis(m-phenoxyphenyl)ether.

12. The method of claim 9, wherein administering includes administering 6-acetyl-5,6,6a,7-tetrahydro-4H-dibezo(de,g)quinoline.

13. The method of claim 9, wherein administering includes administering (+)-N-(p-(2-methylbutoxy)benzylidene)-4-(2-methylbutyl)aniline.

14. The method of claim 9, wherein the at least one compound is derived/extracted from at least one plant species selected from the group consisting of Allium vineale, Salix alba, Agropyrum spp., Petroselinium crispum, Taraxacum officinale, Sesamum indicum, Medicago spp., Piper methysticum, Anthemis spp., Turnera diffusa, Verbascum densiflorum, Ocimum spp., Maranta arundinaceae, Coriandrum sativum, Artemesia dracunculus, Lavendula augustifolia, Mentha pulegium, Centella asiatica, Ginko biloba and Vitis vinifera.

15. The method of claim 9, wherein administering stimulates nitric oxide release in the range of 15 nM to 100 nM in pedal ganglia cells

16. The method of claim 9, wherein administering stimulates nitric oxide release in the range of 50 nM to 100 nM in endothelial cells.

17. The method of claim 9, wherein the individual is suffering from at least one condition selected from a group consisting of: inflammation, bacterial infection, viral infection, and asthma.
